(19) United States
(12) Patent Application Publication
(10) Pub. No.: US 2026/0341178 A1
(43) Pub. Date: Oct. 29, 2026

(54) APPARATUS FOR WARM-UP SMART COACHING

(71) Applicant: Polar Electro Oy, Kempele (FI)

(72) Inventors: Daniela Olstad, Kempele (FI); Elias Pekonen, Kempele (FI); Juhani Kemppainen, Kempele (FI)

(73) Assignee: Polar Electro Oy, Kempele (FI)

(21) Appl. No.: 19/481,966

(22) PCT Filed: May 23, 2024
(86) PCT No.: PCT/FI2024/050263

§ 371 Date: Nov. 6, 2025

(30) Foreign Application Priority Data
May 24, 2023 (GB) 2307763.9

(51) Int. Cl. G09B 19/00; A61B 5/00; A61B 5/024; A61B 5/11; G16H 40/67; G16H 50/30

(52) U.S. Cl. CPC G09B 19/003; A61B 5/02438; A61B 5/1118; A61B 5/681; A61B 5/742; G16H 40/67; G16H 50/30

(57) ABSTRACT

A wearable device for warm-up smart coaching includes at least one sensor configured to perform measurements for an indicator, at least one user interface, and a processing unit. The processing unit is configured to perform at least the following: detecting a start of a warm-up exercise; determining, based on measurement results received from the at least one sensor, whether at least one warm-up criterion for a completion of the warm-up exercise is met; and triggering, in response to the at least one warm-up criterion being met, at least a notification to a user via the at least one user interface, wherein the notification comprises information on the completion of the warm-up exercise.

### CROSS-REFERENCE TO RELATED APPLICATIONS

This application is a national phase application of International Application No. PCT/FI2024/050263, filed May 23, 2024, which claims benefit and priority to Great Britain Application No. 2307763.9, filed May 24, 2023, which are incorporated by reference herein in their entireties.

### BACKGROUND

### Technical Field

The present invention relates to a field of wearable devices.

### SUMMARY

A warm-up exercise before a training session is beneficial for preparing an athlete's body by increasing heart rate, breathing frequency, and blood flow in working muscles. A warmed-up muscle is less inclined to injury. On the other hand, too long or overly intensive warm-up exercise may diminish performance during the training session.

Providing a wearable device configured to give guidance for a suitable warm-up exercise before training may be beneficial.

### BRIEF DESCRIPTION

According to an aspect, there is provided the subject-matter of the independent claims. Embodiments are defined in the dependent claims.

### BRIEF DESCRIPTION OF THE DRAWINGS

In the following, various example embodiments will be described in greater detail with reference to the accompanying drawings, in which

FIG. 1 shows a simplified use case scenario and a schematic block diagram;

FIG. 2 illustrates an exemplary embodiment; and

FIGS. 3 and 4 are flow charts illustrating example functionalities.

### DETAILED DESCRIPTION

The following embodiments are exemplary. Although the specification may refer to “an”, “one”, or “some” embodiment(s) in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments/examples to consist of only those features that have been mentioned and such embodiments may contain also features/structures that have not been specifically mentioned. Further, although terms including ordinal numbers, such as “first”, “second”, etc., may be used for describing various elements, the structural elements are not restricted by the terms. The terms are used merely for the purpose of distinguishing an element from other elements. For example, a first element could be termed a second element, and similarly, a second element could be also termed a first element without departing from the scope of the present disclosure.

Different embodiments and examples are described below using single units, models, equipment, and memory, without restricting the embodiments/examples to such a solution. Concepts called cloud computing and/or virtualization may be used. The virtualization may allow a single physical computing device to host one or more instances of virtual machines that appear and operate as independent computing devices, so that a single physical computing device can create, maintain, delete, or otherwise manage virtual machines in a dynamic manner. It is also possible that device operations will be distributed among a plurality of servers, nodes, devices, or hosts. In cloud computing network devices, computing devices, and/or storage devices provide shared resources. Some other technology advancements, such as Software-Defined Networking (SDN), may cause one or more of the functionalities described below to be migrated to any corresponding abstraction or apparatus or device. Correspondingly, Web 3.0, also known as the third-generation internet, implementing for example blockchain technology, may cause one or more of the functionalities described below to be distributed across a plurality of apparatuses or devices. Therefore, all words and expressions should be interpreted broadly, and they are intended to illustrate, not to restrict, the embodiment.

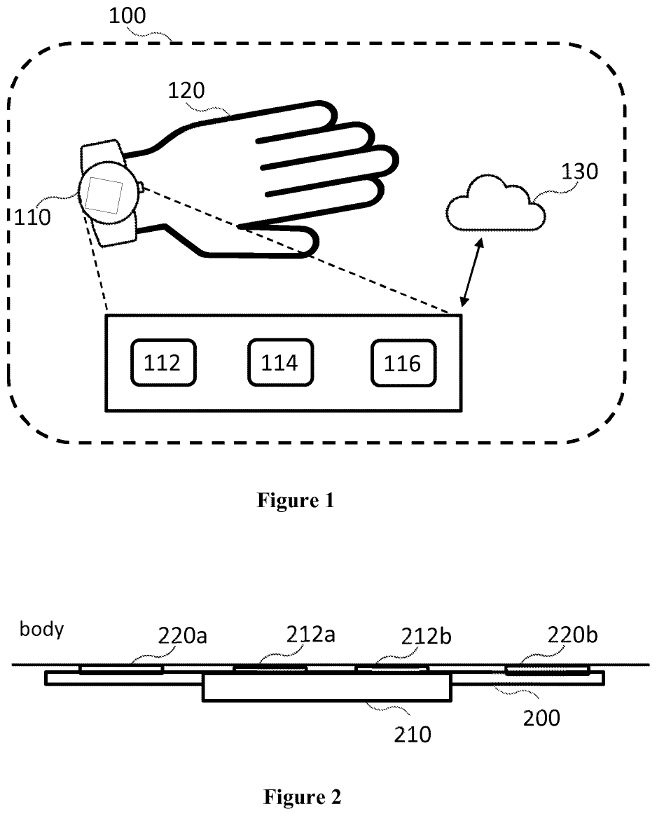

A simplified use case scenario of a wearable device configured to at least perform warm-up monitoring and guidance is illustrated in FIG. 1. FIG. 1 is a schematic block diagram showing only some functional entities, all being logical units whose implementation and/or number may differ from what is shown. The connections shown in FIG. 1 are logical connections; the actual physical connections may be different. It is apparent to a person skilled in the art that the wearable device comprises any number of shown elements, other equipment, other functions, and other structures that are not illustrated. They, as well as the protocols used, are well known by persons skilled in the art and are irrelevant to the actual invention. Therefore, they need not to be discussed in more detail here.

In the example illustrated in FIG. 1, the use case scenario 100 comprises a wearable device 110 wearable by a user 120 against skin, optionally connectable to one or more clouds 130. The wearable device may be, e.g., a smart watch or a smart garment. The wearable device 110 comprises at least one sensor 112, at least one user interface 114, and a processing unit 116. Alternatively, or additionally, the at least one sensor 112 may be comprised in a second wearable device (not shown in FIG. 1) such as a chest strap that is connectable to the wearable device 110. The wearable device 110 may be connectable to the one or more clouds 130 via one or more networks (not shown in FIG. 1). The wearable device 110 may transmit information such as measurement results to the one or more clouds 130 and/or receive information such as training instructions from the one or more clouds 130.

The at least one sensor 112 is configured to perform measurements for an indicator. The at least one sensor 112 may respond to or detect a physiological quantity or a physiological property of the user and output, e.g., an electrical signal corresponding to the physiological quantity or the physiological property of the user. The measurements for the indicator may be values associated with the physiological quantity or the physiological property of the user. The indicator may be understood as representing the physiological quantity or the physiological property of the user. The measurements for the indicator may be utilised, e.g., to determine a completion of the warm-up exercise. The completion of the warm-up exercise may be determined by using at least one warm-up criterion for the completion of the warm-up exercise. The at least one warm-up criterion for the completion of the warm-up exercise may be understood to be met when a measurement value for the indicator meets a pre-determined threshold. Additionally, or alternatively, the measurements for the indicator may be utilised to determine a progress or a status of the warm-up exercise.

The at least one sensor 112 may be a body temperature sensor configured to perform measurements for a body temperature indicator. The body temperature indicator may be, e.g., a body core temperature, a skin temperature, or a body temperature measured from ear. The body core measurement may be measured, e.g., by a telemetry pill measurement using an ingestible telemetric temperature pill configured to perform telemetry measurements. The skin temperature may be measured, e.g., from chest skin or wrist skin using, e.g., a thermal noise measurement with antennas or a micro-electro-mechanical system (MEMS) sensor measurement using a skin-touching patch. Increased body temperature may indicate increased muscle temperature. Increasing muscle temperature by the warm-up exercise may decrease stiffness of muscles and joints, increase release of oxygen from haemoglobin and myoglobin in blood, speed metabolic reactions, and/or increase transmission of nerve impulses.

Alternatively, or additionally, the at least one sensor 112 may be a heart rate sensor configured to perform measurements for a heart rate indicator. The heart rate sensor may be, e.g., a heart activity sensor or an electrocardiography sensor.

Alternatively, or additionally, the at least one sensor 112 may be a sweat sensor configured to perform measurements for a sweat indicator. The sweat sensor may be the electrocardiography sensor. The sweat indicator may be a skin resistance measurement. A value for skin resistance measured from a skin with dry stratum corneum layer, i.e., outermost layer of epidermis, is higher than the value for skin resistance measured from a skin with sweat ducts that are filled with sweat. Further, the value for skin resistance measured from a skin with moist stratum corneum layer, i.e., wherein the sweat has moistened the skin also outside the sweat ducts, is even smaller than the value for skin resistance measured from the skin with sweat ducts that are filled with sweat.

The at least one user interface 114 may be, e.g., a screen, a push button, a loudspeaker, a microphone, or a touch user interface. The at least one user interface 114 is configured to at least give a notification to the user 120. The notification may comprise, e.g., a sound, a text, an image, and/or a number.

The processing unit 116 is configured to at least perform warm-up exercise detection and determination as explained in more detail below. The processing unit 116 may comprise at least one processor and at least one memory storing a computer program comprising program instructions that configure the at least one processor to execute, e.g., embodiments described herein. The memory may further store a configuration database defining parameters for the processor(s). The processing unit 116 is coupled to the at least one sensor 112 by, e.g., a galvanic connection. Sensors of the at least one sensor 112 may each have a separate galvanic connection to the processing unit 116.

The wearable device 110 may further comprise a communication unit (not shown in FIG. 1) configured to transmit measurement data acquired by the wearable device to an external device such as a smart watch, a smart phone, or a personal computer. The external device may be a training computer configured to monitor a physical exercise performed by the user. The communication unit may be a wireless communication unit supporting a wireless communication protocol such as ANT, ANT+, or Bluetooth®, e.g., Bluetooth Smart®). In an embodiment, the processing unit configured to at least enable performing warm-up exercise detection as explained in more detail below may be comprised also in the external device, or only in the external device. The external device may be connectable to the one or more clouds 130 via one or more networks. The external device may receive information such as measurement results from the wearable device 110 and transmit the information to the one or more clouds 130 and/or receive information such as training instructions from the one or more clouds 130 and transmit the information to the wearable device 110.

FIG. 2 is a simplified block diagram illustrating a schematic structure of an embodiment of the wearable device 110 or the second wearable device 200 such as a chest strap comprising the at least one sensor 112.

Referring to FIG. 2, the at least one sensor comprises a body temperature sensor 210 and an electrocardiography sensor. The body temperature sensor 210 may be, e.g., a microwave radiometer system configured to perform thermal noise measurement. In an embodiment, the body temperature sensor 210 comprises at least one antenna 212a, 212b. The at least one antenna 212a, 212b may be, e.g., a spiral microstrip patch antenna.

The electrocardiography sensor is configured to perform electrocardiogram measurements. The electrocardiography sensor comprises at least two skin electrodes 220a, 220b measuring voltage changes on the user's skin surface. The at least two skin electrodes 220a, 220b are arranged such that they enable obtaining signals from both atrial and ventrical sides of the heart.

In an embodiment, the electrocardiography sensor is also configured to perform a resistance measurement between the at least two skin electrodes 220a, 220b. The resistance measurement may be utilised as the sweat indicator.

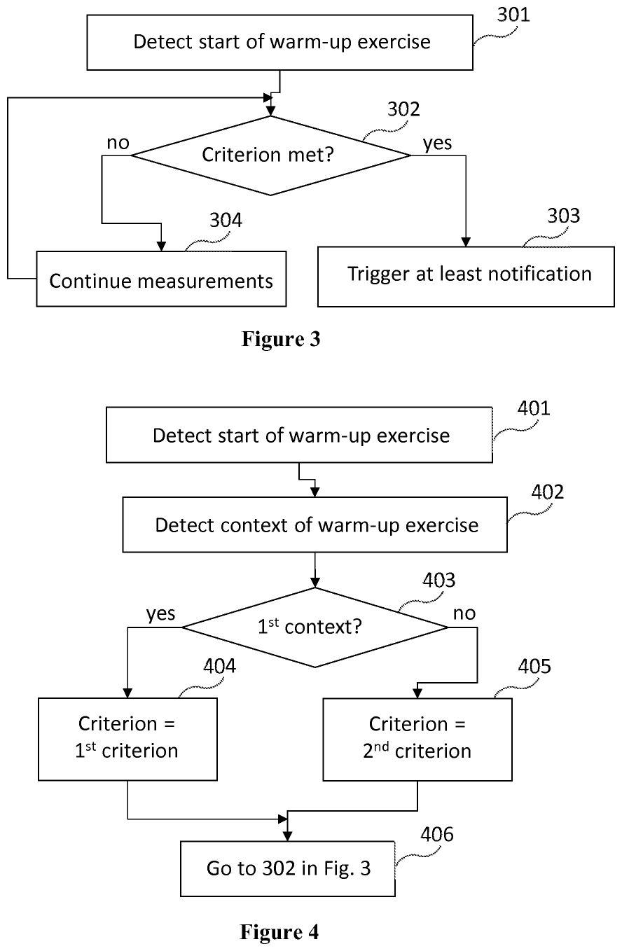

Exemplary functionalities of the processing unit 118 are illustrated in FIG. 3. The at least one sensor is configured to perform measurements for an indicator.

Referring to FIG. 3, a start of a warm-up exercise is detected in block 301. The start of the warm-up exercise may be initiated by the user via the user interface. Alternatively, or additionally, the start of the warm-up exercise may be initiated automatically, e.g., by a motion sensor of the wearable device detecting motion of the user indicating the warm-up exercise. It is then determined in block 302, based on measurement results received from the at least one sensor, whether at least one warm-up criterion for a completion of the warm-up exercise is met. The at least one warm-up criterion may be understood to be met when a measurement value for the indicator meets a pre-determined threshold. If the at least one warm-up criterion is met (block 302: yes), at least a notification to a user via the at least one user interface is triggered in block 303. The notification comprises information on the completion of the warm-up exercise. In an embodiment, also a timestamp for the completion of the warm-up exercise is stored in block 303 on a timeline that is stored in a memory of the wearable device. The timestamp for the completion of the warm-up exercise is, e.g., a sequence of characters or encoded information identifying when the completion of the warm-up exercise occurred. Additionally, other timestamps may be stored in the memory of the wearable device, such as, e.g., a timestamp for the start of the warm-up exercise or timestamps for different phases of the warm-up exercise. In an embodiment, also a start of a training exercise is triggered in block 303. If the at least one warm-up criterion is not met (block 302: no), the measurements for the indicator are continued in block 304 by the at least one sensor.

In an embodiment, one of the at least one sensor is the body temperature sensor configured to perform measurements for the body temperature indicator. One of the at least one warm-up criterion is understood to be met when a value of the body temperature indicator measured meets a pre-determined temperature threshold. That is to say, the warm-up exercise may be determined to be completed when the value of the body temperature indicator measured has increased to meet the pre-determined temperature threshold. The pre-determined temperature threshold may be, e.g., 37.5 °C or 38 °C.

In an embodiment, one of the at least one sensor is the heart rate sensor configured to perform measurements for the heart rate indicator. One of the at least one warm-up criterion is a value of the heart rate indicator measured meeting a pre-determined heart rate threshold. That is to say, the warm-up exercise may be determined to be completed when the value of the heart rate indicator measured has increased to meet the pre-determined heart rate threshold. The pre-determined heart rate threshold may be determined using a pre-determined maximum heart rate of the user. The pre-determined heart rate threshold may be a percent-age of the maximum heart rate of the user, e.g., 60% or 70% of the pre-determined maximum heart rate of the user. The pre-determined maximum heart rate of the user may be determined using, e.g., a fitness test or it may be approximated on the basis of personal information such as, e.g., age of the user.

In an embodiment, one of the at least one sensor is the sweat sensor configured to perform measurements for the sweat indicator. The sweat sensor may be the electrocardiography sensor configured to measure resistance between the at least two skin electrodes. One of the at least one warm-up criterion is a value of the sweat indicator measured decreasing to a pre-determined portion of a reference value. The reference value may be determined by measuring the value of the sweat indicator at the start of the warm-up exercise. The measurement unit for skin resistance is ohm (Ω). Skin resistance of a skin with dry stratum corneum layer may be, e.g., 10 MΩ. Skin resistance of a skin with sweat ducts filled with sweat may be, e.g., 100 kΩ. Skin resistance of a skin with moist stratum corneum may be, e.g., 10 kΩ. In an embodiment, the pre-determined portion of the reference value is one thousandth. The reference value may be determined to be, e.g., 10 MΩ. The warm-up exercise may be determined to be completed when the value of the sweat indicator measured has decreased to meet one thousandth of 10 MΩ, that is, 10 kΩ.

In an embodiment, the wearable device comprises the body temperature sensor configured to perform measurements for the body temperature indicator and the electrocardiogram sensor configured to perform measurements for the heart rate indicator and/or the sweat indicator. When measurements are performed for more than one indicator, the warm-up exercise may be determined to be completed when at least one of the indicator values meets the pre-determined threshold or portion of the reference value as explained above.

Further exemplary functionalities of the processing unit 118 are illustrated in FIG. 4. Intensity of a training exercise that is to follow the warm-up exercise may indicate which warm-up criterion is to be used.

Referring to FIG. 4, a start of a warm-up exercise is detected in block 401. A context of the warm-up exercise is detected in block 402. The context of the warm-up exercise may be determined, e.g., on the basis of a training exercise or a sport mode selected by the user via the user interface. The training exercise may be categorised, e.g., by intensity and/or duration. Thus, the training exercise may be a low intensity training exercise or a high intensity training exercise. Alternatively, the training exercise may be a long training exercise or a short training exercise. Alternatively, the training exercise may be a long low intensity exercise or a short high intensity exercise. The context of the warm-up exercise may be a first context, e.g., low intensity training, or a second context, e.g., high intensity training. It is resolved in block 403 if the context of the warm-up exercise is the first context. If the context is the first context (block 403: yes), one warm-up criterion of the at least one warm-up criterion is determined in block 404 to be a first warm-up criterion. If the context is the second context (block 403: no), one warm-up criterion of the at least one warm-up criterion is determined in block 404 to be a second warm-up criterion. The process then continues to determine in block 302 in FIG. 3, based on measurement results received from the at least one sensor, whether at least one warm-up criterion for a completion of the warm-up exercise is met.

In an embodiment, the context of the warm-up exercise may be determined, e.g., on the basis of user information, such as a training target or fitness information, provided to the processing unit by the user via the user interface.

In an embodiment, one of the at least one sensor is the core temperature sensor configured to perform measurements for the core temperature indicator. The first warm-up criterion is a value of the core temperature indicator measured meeting a first pre-determined temperature threshold and the second warm-up criterion is the value of the core temperature indicator measured meeting a second pre-determined temperature threshold. The first pre-determined temperature threshold is smaller than the second pre-determined temperature threshold. If the context of the exercise is the first context, e.g., the low intensity training, the warm-up exercise may be determined to be completed when the value of the core-temperature indicator measured has increased to meet the first pre-determined temperature threshold. The first pre-determined temperature threshold may be, e.g., 37.5 °C. If the context of the exercise is the second context, e.g., the high intensity training, the warm-up exercise may be determined to be completed when the value of the core-temperature indicator measured has increased to meet the second pre-determined temperature threshold. The second pre-determined temperature threshold may be, e.g., 38 °C.

In an embodiment, one of the at least one sensor is the heart rate sensor configured to perform measurements for the heart rate indicator. The first warm-up criterion is a value of the heart rate indicator measured meeting a first pre-determined heart rate threshold and the second warm-up criterion is the value of the heart rate indicator measured meeting a second pre-determined heart rate threshold. In an embodiment, the first pre-determined heart rate threshold is less than 60% of a maximum heart rate of the user and the second pre-determined heart rate threshold is less than 70% of the maximum heart rate of the user and greater than the first pre-determined heart rate threshold. If the context of the exercise is the first context, e.g., the low intensity training, the warm-up exercise may be determined to be completed when the value of the heart rate indicator measured has increased to meet the first pre-determined heart rate threshold, that is, 60% of the maximum heart rate of the user. If the context of the exercise is the second context, e.g., the high intensity training, the warm-up exercise may be determined to be completed when the value of the heart rate indicator measured has increased to meet the second pre-determined heart rate threshold, that is, 70% of the maximum heart rate of the user.

In an embodiment, the notification to the user via the at least one user interface comprises instructions on a further specific warm-up exercise. Contents of the further specific warm-up exercise are pre-determined, e.g., on the basis of the training exercise that is to follow the warm-up exercise and/or on the basis of an individual physiological constitution of the user. The further specific warm-up exercise may comprise exercises such as, e.g., stretching exercises or mobility exercises, which may be aimed to improve the user's performance in a specific sport. The pre-determined contents of the further specific warm-up exercise may be stored in the at least one memory of the processing unit and/or in the one or more clouds.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), graphics processing units (GPUs), processors, controllers, micro-controllers, microprocessors, other electronic units designed to perform the functions described herein, or a combination thereof. For firmware or software, the implementation can be carried out through modules of at least one chipset (e.g., procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

It will be obvious to a person skilled in the art that, as the technology advances, the inventive concept can be implemented in various ways. The invention and its embodiments are not limited to the examples described above but may vary within the scope of the claims.

What is claimed is:

1. (canceled)

2. (canceled)

3. (canceled)

4. (canceled)

5. (canceled)

6. (canceled)

7. A wearable device comprising:
at least one sensor configured to perform measurements for an indicator, wherein one of the at least one sensor is a heart rate sensor configured to perform measurements for a heart rate indicator, one of the at least one sensor is a sweat sensor configured to perform measurements for a sweat indicator, and one of the at least one sensor is a body temperature sensor configured to perform measurements for a body temperature indicator;
at least one user interface; and
a processing unit configured to perform operations comprising:
detecting a start of a warm-up exercise;
enabling, in response to the detected start of the warm-up exercise, the sweat sensor to measure a reference value of the sweat indicator;
detecting a context of the warm-up exercise, wherein a first context is a low intensity training and a second context is a high intensity training;
determining, in response to the context detected being the first context, one warm-up criterion of at least one warm-up criterion for a completion of the warm-up exercise to be a first warm-up criterion for the heart rate indicator and one warm-up criterion of the at least one warm-up criterion for the completion of the warm-up exercise to be a first warm-up criterion for the body temperature indicator, and determining, in response to the context detected being the second context, one warm-up criterion of the at least one warm-up criterion to be a second warm-up criterion for the heart rate indicator and one warm-up criterion of the at least one warm-up criterion to be a second warm-up criterion for the body temperature indicator;
determining, based on measurement results received from the at least one sensor, whether the at least one warm-up criterion for the completion of the warm-up exercise is met, wherein the first warm-up criterion for the heart rate indicator is a value of the heart rate indicator measured to have increased to meet a first pre-determined heart rate threshold and the second warm-up criterion for the heart rate indicator is the value of the heart rate indicator measured to have increased to meet a second pre-determined heart rate threshold, the first pre-determined heart rate threshold being less than 60 percent of a maximum heart rate of the user and the second pre-determined heart rate threshold being less than 70 percent of the maximum heart rate of the user and greater than the first pre-determined heart rate threshold, wherein, the first warm-up criterion for the body temperature indicator is a value for the body temperature indicator measured to have increased to meet a first pre-determined temperature threshold and the second warm-up criterion for the body temperature indicator is the value of the body temperature indicator measured to have increased to meet a second pre-determined temperature threshold, the first pre-determined temperature threshold being smaller than the second pre-determined temperature threshold, wherein one of the at least one warm-up criterion is a value of the sweat indicator measured decreasing to a pre-determined portion of the reference value of the sweat indicator; and
triggering, in response to the at least one warm-up criterion being met, at least a notification to a user via the at least one user interface, wherein the notification comprises information on the completion of the warm-up exercise.

8. The wearable device of claim 7, wherein the processing unit is further configured to perform, in response to the at least one warm-up criterion being met, at least one of operation comprising:
storing a timestamp for completion of the warm-up exercise on a timeline stored in a memory of the wearable device; and
triggering a start of a training exercise.

9. The wearable device of claim 7, wherein the notification to the user comprises instructions for a further specific warm-up exercise.

10. A computer-implemented method, the method comprising:
performing measurements for an indicator with at least one sensor, wherein one of the at least one sensor is a heart rate sensor configured to perform measurements for a heart rate indicator, one of the at least one sensor is a sweat sensor configured to perform measurements for a sweat indicator, and one of the at least one sensor is a body temperature sensor configured to perform measurements for a body temperature indicator;
detecting a start of a warm-up exercise;
enabling, in response to the detected start of the warm-up exercise, the sweat sensor to measure a reference value of the sweat indicator;
detecting a context of the warm-up exercise, wherein a first context is a low intensity training and a second context is a high intensity training;
determining, in response to the context detected being the first context, one warm-up criterion of at least one warm-up criterion for a completion of the warm-up exercise to be a first warm-up criterion for the heart rate indicator and one warm-up criterion of the at least one warm-up criterion for the completion of the warm-up exercise to be a first warm-up criterion for the body temperature indicator, and determining, in response to the context detected being a second context, one warm-up criterion of the at least one warm-up criterion to be a second warm-up criterion for the heart rate indicator and one warm-up criterion of the at least one warm-up criterion to be a second warm-up criterion for the body temperature indicator;
determining, based on measurement results received from at least one sensor, whether the at least one warm-up criterion for the completion of the warm-up exercise is met, wherein the first warm-up criterion for the heart rate indicator is a value of the heart rate indicator measured to have increased to meet a first pre-determined heart rate threshold and the second warm-up criterion for the heart rate indicator is the value of the heart rate indicator measured to have increased to meet a second pre-determined heart rate threshold, the first pre-determined heart rate threshold being less than 60 percent of a maximum heart rate of the user and the second pre-determined heart rate threshold being less than 70 percent of the maximum heart rate of the user and greater than the first pre-determined heart rate threshold, wherein, the first warm-up criterion for the body temperature indicator is a value for the body temperature indicator measured to have increased to meet a first pre-determined temperature threshold and the second warm-up criterion for the body temperature indicator is the value of the body temperature indicator measured to have increased to meet a second pre-determined temperature threshold, the first pre-determined temperature threshold being smaller than the second pre-determined temperature threshold, wherein one of the at least one warm-up criterion is a value of the sweat indicator measured decreasing to a pre-determined portion of the reference value of the sweat indicator; and
triggering, in response to the at least one warm-up criterion being met, at least a notification to a user via at least one user interface, wherein the notification comprises information on the completion of the warm-up exercise.

11. A processing unit for a wearable device, the wearable device comprising:
at least one sensor configured to perform measurements for an indicator, wherein one of the at least one sensor is a heart rate sensor configured to perform measurements for a heart rate indicator, one of the at least one sensor is a sweat sensor configured to perform measurements for a sweat indicator, and one of the at least one sensor is a body temperature sensor configured to perform measurements for a body temperature indicator;
at least one processor; and
at least one memory storing a computer program comprising program instructions that, when executed by the at least one processor, cause the processing unit to perform operations comprising:
detecting a start of a warm-up exercise;
enabling, in response to the detected start of the warm-up exercise, the sweat sensor to measure a reference value of the sweat indicator;
detecting a context of the warm-up exercise, wherein a first context is a low intensity training and a second context is a high intensity training;
determining, in response to the context detected being the first context, one warm-up criterion of at least one warm-up criterion for a completion of the warm-up exercise to be a first warm-up criterion for the heart rate indicator and one warm-up criterion of the at least one warm-up criterion for the completion of the warm-up exercise to be a first warm-up criterion for the body temperature indicator, and determining, in response to the context detected being the second context, one warm-up criterion of the at least one warm-up criterion to be a second warm-up criterion for the heart rate indicator and one warm-up criterion of the at least one warm-up criterion to be a second warm-up criterion for the body temperature indicator;
determining, based on measurement results received from the at least one sensor, whether the at least one warm-up criterion for the completion of the warm-up exercise is met, wherein the first warm-up criterion for the heart rate indicator is a value of the heart rate indicator measured to have increased to meet a first pre-determined heart rate threshold and the second warm-up criterion for the heart rate indicator is the value of the heart rate indicator measured to have increased to meet a second pre-determined heart rate threshold, the first pre-determined heart rate threshold being less than 60 percent of a maximum heart rate of the user and the second pre-determined heart rate threshold being less than 70 percent of the maximum heart rate of the user and greater than the first pre-determined heart rate threshold, wherein, the first warm-up criterion for the body temperature indicator is a value for the body temperature indicator measured to have increased to meet a first pre-determined temperature threshold and the second warm-up criterion for the body temperature indicator is the value of the body temperature indicator measured to have increased to meet a second pre-determined temperature threshold, the first pre-determined temperature threshold being smaller than the second pre-determined temperature threshold, wherein one of the at least one warm-up criterion is a value of the sweat indicator measured decreasing to a pre-determined portion of the reference value of the sweat indicator; and
triggering, in response to the at least one warm-up criterion being met, at least a notification to a user via at least one user interface of the wearable device, wherein the notification comprises information on the completion of the warm-up exercise.

12. The processing unit of claim 11, wherein the program instructions, when executed by the at least one processor further cause the processing unit, in response to the at least one warm-up criterion being met, to perform operations comprising:
storing a timestamp for completion of the warm-up exercise on a timeline stored in a memory of the wearable device; and
triggering a start of a training exercise.

13. The wearable device of claim 8, wherein the notification to the user comprises instructions for a further specific warm-up exercise.
